(19) United States
(12) Patent Application Publication
(10) Pub. No.: US 2010/0196443 A1
(43) Pub. Date: Aug. 5, 2010

(54) USE OF CORDYCEPS SPECIES OR EXTRACT THEREOF IN THE EGG PRODUCTION

(75) Inventor: Yue Xu, Singapore (SG)

Correspondence Address: HESLIN ROTHENBERG FARLEY & MESITI PC, 5 COLUMBIA CIRCLE, ALBANY, NY, 12203, US

(73) Assignee: A P NUTRIPHARM PTE. LTD., Singapore (SG)

(21) Appl. No.: 12/670,546

(22) PCT Filed: Jul. 4, 2008
(86) PCT No.: PCT/SG08/00239

§ 371 Date: Jan. 25, 2010

(30) Foreign Application Priority Data
Jul. 25, 2007 (SG) 200705352-3

(51) Int. Cl. A61K 9/00; A61K 36/06; A61K 31/52; A61P 11/00; A61P 3/10; A61P 35/00; A61P 13/12

(52) U.S. Cl. 424/439; 424/195.15; 514/263.23

(57) ABSTRACT

A dietary supplement or animal feed includes at least one Cordyceps species or extract thereof. The dietary supplement and/or animal feed is preferably for oviparous animals and in particular for the production of eggs.

### FIELD OF THE INVENTION

The present invention relates to Cordyceps extract and uses thereof. In particular, the invention relates to a dietary supplement comprising extract(s) of Cordyceps species. The dietary supplement may be added to feed and/or drinking liquid to improve the health and general well being of animals, particularly for oviparous animals. The invention also relates to the use of the dietary supplement in animal feed and/or drinking liquid to improve the quality of eggs produced.

### BACKGROUND OF THE INVENTION

Animal husbandry is the agricultural practice of breeding and rearing livestock and has a long history. The field of animal husbandry includes the management, handling, nutrition, breeding and welfare of animals. Animals are cultivated for meat and for produce such as wool, milk and eggs. Animals that are commonly reared for these purposes include cattle, goats, sheep, pigs, fowl and poultry.

Research into animal nutrition and animal feed formulation aims to improve the growth, health and wellbeing of animals and the quality of the animal produce. Such animal feed formulations can include complete feeds comprising all the required nutrients, concentrates that provide part of the required nutrients, (such as protein or carbohydrates) or supplements that provide additional micro-nutrients, (for example, vitamins and minerals). The concentrates and supplements can be purchased as separate formulations by the farmer and mixed into animal feed as necessary.

Poultry, in particular fowl, are very important agricultural animals which are reared as food and for their eggs. These most typically are members of the orders Galliformes (such as chickens and turkeys), and Anseriformes (waterfowl such as ducks and geese). Other than rearing poultry for their meat, eggs are an important poultry produce which can be eaten.

Eggs are very nutritious and contain a large quantity of protein and also vitamins and minerals, including vitamin A, riboflavin, folic acid, vitamin B6, vitamin B12, choline, iron, calcium, phosphorus and potassium. However, the egg yolk contains a significant amount of cholesterol, more than two thirds of the recommended daily cholesterol intake. It is the high cholesterol content in eggs and the associated adverse publicity that poses a challenge to egg producers.

Cholesterol is an important precursor in the synthesis of the sex steroid and adrenal hormones and plays a central role in many biochemical processes. The human body is capable of synthesising cholesterol de novo, but cholesterol is also consumed in the diet. There is link between high cholesterol levels and cardiovascular diseases, and consumers tend to be concerned about cholesterol in the diet.

It is therefore desirable to have a method of producing eggs with a reduced cholesterol content for the purposes of catering to consumer satisfaction. It would also be beneficial to improve the nutrients of eggs.

### SUMMARY OF THE INVENTION

According to a first aspect, the present invention provides a dietary supplement comprising at least one Cordyceps species or at least one extract of at least one Cordyceps species. In general, any Cordyceps species is contemplated. The Cordyceps species may be selected from the group consisting of Cordyceps sinensis, Cordyceps militaris, Cordyceps gracilis, Cordyceps ophioglossoides, Cordyceps memorabiffis, Cordyceps sphecocephala, Cordyceps hawkesii, Cordyceps martialis, Cordyceps sobolifera and Cordyceps capitata. The Cordyceps extract may be in the form of a powder or a paste.

According to another aspect, the invention provides a method of supplementing the diet of animals with at least one Cordyceps species or extract thereof. Preferably, the dietary supplement comprising the extract is mixed with feed and/or drinking liquid and made available to the animal. The animal is preferably an oviparous animal and the method is preferably for producing at least one egg. More preferably, the oviparous animal is poultry. Most preferably, the poultry is fowl. Preferably, the dietary supplement is mixed with the feed and provided to the animal.

According to another aspect, the invention provides drinking liquid supplemented with a dietary supplement comprising at least one extract of at least one Cordyceps species.

According to a further aspect, the invention provides an animal feed comprising at least one feed ingredient and at least one extract of at least one Cordyceps species. The Cordyceps species may be selected from the group consisting of Cordyceps sinensis, Cordyceps militaris, Cordyceps gracilis, Cordyceps ophioglossoides, Cordyceps memorabillis, Cordyceps sphecocephala, Cordyceps hawkesii, Cordyceps martialis, Cordyceps sobolifera and Cordyceps capitata. The Cordyceps extract may be a powder or a paste. Any of the main feed ingredients may be used for preparing the animal feed, including corn, soybeans, sorghum, oats and barley.

The invention also provides a method of feeding at least one animal with the animal feed described above. The animal is preferably an oviparous animal. More preferably, the animal is poultry. Most preferably, the animal is fowl. In addition, the method of feeding is for producing eggs.

According to another aspect, the invention also relates to an animal that is fed by the method as described and/or whose diet is supplemented as described. In particular, the animal is for producing meat. The invention therefore also relates to the meat from an animal that is fed by the method as described and/or whose diet is supplemented as described. In particular the meat may be processed meat and/or meat from the carcass of the animal. In particular, the meat may be processed meat, isolated meat and/or meat from the carcass of the animal.

In another aspect, the invention provides a method of producing at least one egg comprising supplementing the diet of at least one animal with at least one extract of at least one Cordyceps species and/or feeding the animal with an animal feed comprising at least one feed ingredient and at least one extract of at least one Cordyceps species.

In another aspect, the invention relates to the use of at least one extract of at least one Cordyceps species in the preparation of a dietary supplement for producing at least one egg.

According to another aspect, the invention relates to an egg obtainable according to any of the methods of the invention.

### BRIEF DESCRIPTION OF THE FIGURES

FIG. 1 shows the HPLC profiles of (A) whole eggs produced by layers fed animal feed comprising Cordyceps sinensis extract (B) eggs produced by control layers (C) cordycepin standard (D) egg yolks from eggs produced by layers fed animal feed comprising Cordyceps sinensis extract and (E) egg whites from eggs produced by layers fed animal comprising Cordyceps sinensis extract.

FIG. 2 shows a comparison of Three-Yellow chickens fed on a diet supplemented with Cordyceps sinesnsis mycelium powder (test group) and control chickens. (A) and (B) show two chickens from the test group, (C) and (D) show two chickens from the control group.

### DEFINITIONS

Extract includes a product produced by extracting from the original material, an essence from the original material, a concentrate from the original material, an emulsion of the original material, a product produced by drying, grinding or compressing the original material, such as into a powder, a cake, or any product or material derived from the original material, including a paste or dregs.

Oviparous animals refer to animals which lay eggs, with little or no embryonic development within the parent. Oviparous animals comprise many fish, amphibians and reptiles, all birds, the monotremes (mammals which lay eggs), and most insects and arachnids.

Poultry refers to any kind of domesticated bird or bird captive-raised for meat or eggs; ostriches for example are sometimes kept as poultry.

Fowl refers to certain birds often used as food by humans. It includes some poultry such as chickens or turkeys, game birds such as pheasants or partriges, other wildfowllike guineafowl of peafowl, and waterfowl such as ducks or geese. The birds referred to as “fowl” belong to one of two orders, namely the gamefowl or landfowl (Galliformes) and the waterfowl (Anseriformes).

Animal feed refers to substances for feeding animals, whether solid or liquid or a paste and include compound feed blended from raw materials, fodder and liquids for drinking, such as water.

As used herein, predominantly refers to more than 50%, in particular, more than 60%, more than 70%, more than 80%, more than 90%, or more than 99%.

### DETAILED DESCRIPTION OF THE INVENTION

For the invention described herein, the at least one extract of at least one Cordyceps species for the dietary supplement and/or the animal feed may be derived from naturally occurring Cordyceps species. Naturally occurring Cordyceps strains grow on the body of a host caterpillar or insect larvae, which becomes mummified ay an internal mat of Cordyceps mycelium. Eventually, a large, clavate stroma, sometimes as much as 10-15 cm in length grows out from the mummified host and bears an immersed perithecia in its upper part. Wild Cordyceps strains are very expensive and can possess high concentrations of heavy metals and also carry microbial contamination. The Cordyceps extract used may be derived from Cordyceps species grown in large-scale culture. In particular, the Cordyceps extract used may be prepared according to the method disclosed in the Singapore patent application SG 200400686-2 (SG patent 129276), the whole content of which is herein incorporated by reference. However, any other method known in the art for preparing Cordyceps extract useful for the purpose of the present invention may also be used.

The dietary supplement and/or animal feed comprising the dietary supplement of the invention is beneficial when given to the animal. It will enhance the health and wellbeing of the animal. The Cordyceps extract in the dietary supplement and/or animal feed can also stimulate the immune system of poultry, providing improved resistance to diseases including but not limited to fowl cholera, avian influenza including the H5N₁ strain, Newcastle disease and/or Marek's disease.

The dietary supplement and/or animal feed may be used both for barn, free range and/or high intensity type rearing of oviparous animals. The dietary supplement and/or animal feed comprising an extract of at least one Cordyceps species may be an important advantage in increasing the immunity and reducing susceptibility to diseases, especially in high intensity type rearing of animals.

\The present inventors have also surprisingly found that the method described herein results in the production of eggs with reduced cholesterol content. The cholesterol content of chicken eggs is reported to range from 368.20±22.54 to 437.63±24.61 mg cholesterol per 100 g of fresh egg matter (Vorlová et al., 2001). The eggs produced by the method of the present invention comprise less than 350 mg cholesterol per 100 g of egg matter. In particular, the eggs produced by the method of the present invention comprise less than 300 mg cholesterol per 100 mg of egg matter. More specifically, the eggs of the present invention comprise 202 to 293 mg cholesterol per 100 g of egg matter. Accordingly, the eggs of the present invention comprise reduced cholesterol content compared to at least one egg not produced by the method of the present invention.

Therefore, according to another aspect, the invention provides an egg comprising less than 350 mg cholesterol per 100 g of egg matter. In particular, the invention provides an egg comprising less than 300 mg cholesterol per 100 g of egg matter.

Further, the present inventors have surprisingly found that the method and use described above results in the transfer of cordycepin into the eggs produced. It has been found that the eggs produced each comprises 006-0.08 g of cordycepin depending on the amount of dietary supplement provided to the animal. The cordycepin is predominantly in the egg yolk.

Therefore, according to another aspect, the invention provides an egg comprising cordycepin. In particular, the invention provides an egg comprising 0.006 to 0.08 mg cordycepin per 100 of egg matter.

Cordycepin (3-deoxyadenosine) is a derivative of the nucleoside adenosine produced by Cordyceps species. Cordycepin is often used as a biological marker for Cordyceps species. In this instance, the presence of cordycepin in the eggs produced suggests that other active and/or beneficial ingredients of the Cordyceps extract are also transferred into the eggs.

The following pharmacological effects of Cordyceps have been studied by either animal models and/or human (clinical) trials:
IMMUNOMODULATION: Cordyceps sinensis (CS) can significantly enhance the immunity of the body.
ANTI-CANCER AND ANTI-MICROBIAL EFFECTS: Associated with immunomodulatory function, CS shows an anti-cancer effect, and was reported to inhibit viral, bacterial and fungal infections.
ANTIDIABETIC ACTIVITY: Polysaccharide in CS was reported to significantly lower blood glucose levels.
ENERGY METABOLISM AND PERFORMANCE ENHANCEMENT: CS was reported to stimulate hepatic energy metabolism.
RESPIRATORY AND PULMONARY FUNCTIONS: CS was reported to relax the bronchial walls as well as having sedative, antitussive, and anti-inflammatory properties, thus it is effective against cough, phlegm, asthma and other respiratory ailments.
CHOLESTEROL AND LIPID METABOLISM: The report indicated plasma triglyceride, “bad cholesterol” such as LDL-c and vLDL-c, and total cholesterol levels were lowered by CS.
EFFECTIVE ON RENAL DISORDER: There are indications that CS may be of use in the treatment of Berger's disease (IgA nephropathy).
EFFECTIVE ON SEXUAL DYSFUNCTIONS AND REPRODUCTIVE DISORDER: Clinical improvement of impotence by taking CS was reported.
OTHERS: The reports suggested that CS can stimulate erythropoiesis in mouse bone marrow, and inhibits platelet aggregation.

In addition, the compound cordycepin has been extensively studied and has been shown to have anti-tumour and antifungal properties. In one study, it was shown that cordycepin can inhibit nucleolar RNA synthesis in RNA cell (Siev et al., 1969)

Accordingly, another aspect of the invention provides a method of treating and or prophylaxis of at least one disease comprising administering at least one egg as described herein, an egg extract or egg product from at least one egg as described herein.

The invention also relates to the use of at least one egg as described herein, egg extract or egg product from at least one egg as described herein for the preparation of a composition for the treatment and/or prophylaxis at least one disease. The disease includes respiratory disease, kidney disease, renal disease, diabetes, cancer, tumour and/or infectious disease.

Having now generally described the invention, the same will be more readily understood through reference to the following examples which are provided by way of illustration, and are not intended to be limiting of the present invention.

### Example 1

The extract of at least one Cordyceps species is preferably obtained from Cordyceps cultured according to the method described in published Singapore patent application No. 200400686-2 (SG patent 129276). Cordyceps strains with an FIRDI number are available from the Food Industry Research and Development Institute, Taiwan.

Methods are described below of the production of various strains of Cordyceps. It should be noted that the parameters given for the conditions of growth such as fermentation period, temperature, stirring speed, aeration rate, pH rage etc, are not necessarily limited to the values as shown but may vary within a reasonable degree as is typically understood by people skilled in the art.

Cultivation of Cordyceps sinensis in Fermentation Device

A liquid culture medium comprising a carbon source, a nitrogen source, vitamins, minerals and other growth factors from raw materials of glucose, maltose or powder, yeast extract, and food grade chemicals as listed in Table 1 may be used. In order to produce the Cordyceps, for example, C. sinensis, an isolated strain was first subjected to solid medium culture. The strain C. sinensis APN 4-1 (FIRDI-36421), was grown on and/or kept on yeast malt (YM) slant or plate agar medium (Table 2) or the potato dextrose agar (PDA) medium (Table 3) in test tubes or agar plates at 25° C. for 3 days.

For the first seed culture, shake culture making use of Erlenmeyer flasks was used. One litre of the broth medium (Table 4, or Table 5) contained in each 5 litre conical flask was sterilised at 121° C. for 20 minutes and inoculated with 3-6 slant or plate culture of the C. sinensis APN 4-1 and were then grown under the following conditions:
pH 5.5 (adjusted before inoculation)
25° C.
the flasks rotated on shakers at a speed of about 150 rpm
culture period of 48 hr.

For the second seed culture of C. sinensis, 75 litre (or bigger) stirred bio-reactors (BioLafitte) each containing 50 litres or more of the fermentation medium (Table 1) were sterilised at 121° C. for 30 minutes and then inoculated with 2 litres of the cells from the 1st seed culture (4% of inoculum). The following growth conditions were maintained:
pH 5.5
25° C.
aeration rate of 1:0.2-1:0.9 v/v/m (1500-2700 l/hr for 50 litres of medium)
stirring speed 200 rpm
cultivation period 48 hr

For the cultivation of mycelia of C. sinensis, 800 litre (or a bigger) stirred-tank reactors (for example, BioLafitte), each containing a volume of 600 litres or more of the fermentation medium (Table 1) are inoculated with 50 litres or more of 2nd seeding culture (8% of inoculum). The tanks and the fermentation medium were sterilised at 121° C. for 30 minutes before inoculation.
pH 5.5
25-29° C.
stirring speed 200 rpm
typical aeration rate of 1:0.5-1:1 v/v/m (18-36.0 m3/hr for 600 litre medium)
cultivation period 48-72 hr

When the mycelium had reached the end of its exponential phase or had multiplied enough for the purpose of production, the bottom valves of the 800 litre bio-reactors were opened, and the mycelium of C. sinensis was collected and filtered on vacuum filter or filter pressed; up to 100 kg of mycelium cake with 30% of dry matter was obtained from each bio-reactor. This cake was then dried on a double-cone rotary vacuum dryer or by freeze-drying and a filamentous flour with 27-28% of protein was obtained. The filamentous flour was grounded by a miller to a particle size of 100-200 mesh. The obtained powder was then packaged into aluminium bags.

TABLE 1
Liquid Medium
New medium (g/L) The preferred value is indicated In parentheses

Glucose, 30-50 (40)
Maltose, 30-60 (40)
KH₂PO₄, 0.05-2.0 (1.5)
MgSO₄, 0.05-1.0 (0.3)
Yeast extract, 15-30 (20)

TABLE 2
YM slant/plate agar medium

| Ingredient | Content | |
|---|---|---|
| Yeast extract | 3 | g |
| Malt extract | 3 | g |
| Peptone | 5 | g |
| Dextrose | 10 | g |
| Agar | 20 | g |
| Distilled water | Up to 1 | litre |

TABLE 3
Potato dextrose agar medium

| Ingredients | Content | |
|---|---|---|
| Potato extract | 4.0 | g |
| Glucose | 20.0 | g |
| Agar | 15.0 | g |
| pH | 5.6 | |
| Distilled water | Up to 1 | litre |

TABLE 4
Flask medium-I

| Ingredients | Content g/l | |
|---|---|---|
| Glucose | 40.0 | |
| Maltose powder | 20.0 | g |
| Potato dextrose broth | 24.0 | g |
| Yeast extract | 10.0 | |
| Water | Up to 1 | litre |

TABLE 5
Flask medium-2

| Ingredients | Content g/l | |
|---|---|---|
| Glucose | 40.0 | |
| Maltose powder | 40.0 | |
| KH₂PO₄ | 1.5 | |
| MgSO₄•7H₂0 | 0.3 | |
| Yeast extract/peptone | 20.0 | |
| Water | Up to 1 | litre |

Cultivation of Cordyceps gracilis, Cordyceps memorabillis, and Cordyceps ophioglossoides in Fermentation Device

The strains Cordyceps gracilis APN 4-2 (FIRDI 32217), Cordyceps memorabillis, APN 4-3 (FIRDI 32218) and Cordyceps ophioglossoides, were grown and kept on YM slant or plate agar medium (Table 2) at 25° C. for 3-5 days. The slant cultures of C. gracilis, C. memorabillis or C. ophioglossoides were used to inoculate 50 ml of growth medium (described in Table 5, 6) in 300-ml Erlenmeyer flasks. The mycelia were subjected to shake culture at 25° C. for 72 hr on a rotating shaker at 150 rpm.

For commercial production of C. gracilis, C. memorabillis, and C. ophioglossoides, the multi-stage fermentation as described for Cordyceps sinensis above was used.

Cordyceps militaris

It was found that this strain would not grow well at a temperature higher than 25° C. and also would not grow well on the agar slant/plate. By addition of 0.6% NaNO₃ or 0.01% Riboflavin (vitamin B2) to the YM agar medium (control, Table 2), the cultivation time was shortened to 3 days (grown on the fortified medium) from 7 days. Cordyceps militaris APN 4-5 (FIRDI 32219) was grown and kept on fortified YM medium at 17-23° C., preferably at 20° C. for 3-days before using it for inoculation of the multi-stage fermentation in Example 3.

The culture medium (Table 1) for the cultivation for all other strains of Cordyceps, therefore, differs slightly from that used specifically for Cordyceps militaris (Table 6). For all other strains of Cordyceps, KH₂PO₄ and MgSO₄.7H₂O were used as the mineral source, and for Cordyceps militaris NaNO₃ (Table 3) was used instead.

The hot water extraction which increases the amount of active components recoverable from the medicinal fungi. The water used may also be mixed with a pH buffer, adjusting it to about pH 10.

TABLE 6
Broth Medium for Cordyceps militaris

| Ingredients | Content, g/l | |
|---|---|---|
| Glucose | 40 | g |
| Maltose powder | 40 | g |
| NaNO₃ | 2 | g |
| Yeast extract/peptone | 20 | g |
| Water | Up to 1 | litre |

### Example 2

### Extraction Process of Cordyceps Bio-Active Ingredients

According to conventional methods known in the art, the active ingredients of Cordyceps are extracted for water-soluble and ethanol-soluble compounds. However, these conventional methods of extraction do not include hot water extraction, which is provided in this method and is described below. The absence of a hot water extraction step in conventional methods makes them ineffective in extracting the water-soluble polysaccharides. The hot water may also be buffered at pH 10 for further efficacy.

About 1700 g of wet Cordyceps mycelia were soaked, with gentle agitation, with about 6 litres of hot water (about 90° C.) overnight, i.e. about 24 hrs or more. The Cordyceps were then filtered and the filtrate was then mixed with 2 volumes of 99% ethanol (at 6 litres per volume). The precipitate resulting from the addition of the ethanol to the filtrate was then collected and washed in 50% ethanol, and subsequently lyophilised, obtaining 2.9 g of Cordyceps polysaccharides.

The residue was then extracted for active compounds for a further 4 times by using about 4 litres of 80% ethanol solution, with gentle agitation. The ethanol was then evaporated and the resultant concentrate was freeze-dried, obtaining 101 g of solids. The use of any other solvent other than methanol, acetone, will be evident to any skilled in the art.

The concentrate is in the form of a powder and may be used as the dietary supplement. The specifications of the powder are shown in Table 7.

TABLE 7
Product specifications of powdered Cordyceps obtained by the
method provided

| | |
|---|---|
| Product description | Powdered Cordyceps mycelia, |
| | produced by submerged |
| | cultivation, followed by drying, |
| | grinding And sterilization |
| Product fingerprints | |
| Proteins/glycoproteins: | SDS-PAGE and western blotting |
| Overall: | Fourier transform infrared |
| | (FTIR)spectroscopy |
| Physical appearance: | Powder with good flow property |
| Colour: | Brown or light brown |
| Odour: | Slightly fermented |
| Particle size: | 100 to 200 mesh, |
| Flowing property: | Free-flowing |
| pH (in 10% solution): | 4.5-5.5 |
| Moisture %: | Less than 5% |
| Active components: | |
| Total protein: | 27-28% |
| Total water soluble polysaccharides: | 20% or above |
| Cordycepic acid (mannitol): | 5% or above |
| Cordycepin (3'-deoxyadenosine): | 0.1% or above |
| Adenosine: | 0.15% or above |
| Ash: | Less than 5% |
| Heavy metal: | |
| Arsenic | <0.01 ppm |
| Copper | <15 ppm |
| Lead | <0.2 ppm |
| Mercury | <0.01 ppm |
| Microbial limit: | In compliance with the Singapore |
| Total bacterial count, CFU/g | Medicines Act |
| Total yeast & mould, CFU/g | <10² |
| Total E. coli, per gram | <10² |
| Staphylococcus aureus, per gram | Absent |
| Salmonella spp., per gram | Absent |
| | Absent |

### Example 3

### Preparation of Paste Form of Cordyceps

Upon completion of the large-scale cultivation methods described in Example 1, the mycelium, instead of being collected and dried, was collected by filtering it through a needle-felt polyester filter medium by gravity or by a controlled vacuum force to make the wet mycelium contain 10-20% of total solids. The wet mycelium was then washed twice by water to remove the residues of the medium.

The yield of the Cordyceps paste obtained ranged 300-500 kg per cubic meter of fermentation broth.

The wet mycelium with 10-20% of total solids was subjected to a homogenizing process by either a colloidal mill, or by a homogeniser, or by any type of proper milling machine. A particle size of 0.5-3.0 μm of the wet mycelium was obtained by homogenization. This particle size allowed the wet mycelium to be well dispersed in the solutions. The obtained Cordyceps preparation is indicated as “paste Cordyceps”.

The specific characteristics of the obtained “paste Cordyceps sinensis” are as shown in Table 8.

Different characteristics of other species or strains, other than Cordyceps sinensis (C. sinensis) have also been observed. These characteristics are shown in Table 9.

TABLE 8
Product specification of Cordyceps sinensis in paste form

| | |
|---|---|
| Product description | Cordyceps sinensis mycelia |
| | produced by submerged cultivation, |
| | followed by filtration, packaging and |
| | retorting at 110-121° C. |
| Packaging form | Paste form of products, quantity in |
| | 3 kg or customised size, packed in |
| | laminated retort pouch. |
| Product fingerprints | SDS-PAGE and western |
| Proteins/glycoproteins: | blotting |
| Overall: | Fourier transform infrared |
| | (FTIR) spectroscopy |
| Physical appearance: | |
| Colour: | Brown or light brown |
| Odour: | Slightly fermented |
| Property: | Paste |
| pH (in 10% solution): | 4.5-5.5 |
| Total solid %: | 10-20% |
| Components (dry basis): | |
| Total protein: | 27-28% |
| Total water soluble | 20% or above |
| polysaccharides: | 5% or above |
| Cordycepic acid (mannitol): | 0.1% or above |
| Cordycepin (3'-deoxyadenosine): | 0.15% or above |
| Adenosine: | |
| Ash: | Less than 5% |
| Heavy metal: | In compliance with the Singapore |
| | Medicines Act |
| Arsenic | <0.01 ppm |
| Copper | <15 ppm |
| Lead | <0.2 ppm |
| Mercury | <0.01 ppm |
| Microbial limit: | In compliance with the Singapore |
| | Medicines Act |
| Total bacterial count, CFU/g | <10² |
| Total yeast & mould, CFU/g | <10² |
| Total E. coli, per gram | Absent |
| Staphylococcus aureus, per gram | Absent |
| Salmonella spp., per gram | Absent |

TABLE 9
Characteristics of other Cordyceps strains

| | |
|---|---|
| Product and cell description | 1) C. militaris shows the whitest and |
| | finest mycelia visually, while |
| | C. sinensis is the most coarse. |
| | 2) The paste of C. militaris is more |
| | like milk flavour |
| | 3) The taste of powdered C. militaris |
| | is the sweetest, that of |
| | C. ophioglossoides a little sour, |
| | and the bitter taste of C. sinensis. |
| | 4) Filtered bio-mass of C. militaris |
| | mycelia is more compact than that |
| | of C. sinensis & C. ophioglossoides. |
| | 5) Under microscopy, two spores are |
| | contained inside the ascus of |
| | C. militaris, as comparison with |
| | that of C. sinensis, only one spore |
| | (see photos) |
| FTIR fingerprints | Fingerprints of five species of |
| | Cordyceps in Fourier transform |
| | infrared (FTIR) spectroscopy are very |
| | different, which can be provided if |
| | required. |
| Active compound: Cordycepic acid | Contents of C. militaris & |
| | C. ophioglossoides are higher than |
| | 9%, while C. sinensis is 5-7% only. |
| Active compound: Cordycepin | The content of C. militaris is double |
| | or triple higher than that of |
| | C. sinensis. |
| | But existence of Cordycepin in |
| | C. ophioglossoides is not yet |
| | confirmed. |
| Active compound: Adenosine: | Higher in C. militaris and |
| | C. ophioglossoides than C. sinensis. |
| Active compound: Ophiocordin | A potent protein kinase inhibitor, is |
| (balanol) | contained in C. ophioglossoides only. |

### Example 4

### Preparation of Feed

The animal feed was prepared by mixing 50 g, 125 g or 300 g of the Cordyceps sinensis powder obtained from Example 2 above per ton of commercial or common laying feed.

Alternatively, the powder may be added to the drinking liquid or the paste may be used instead.

### Example 5

### Feeding of Chickens and Properties of Eggs Produced

8000 Bovans Goldline Brown layers were fed the animal feed comprising Cordyceps sinensis prepared as described in Example 4 from 60 weeks to 90 weeks.

During this time, the eggs produced by the layers were sampled and analysed for cordycepin and cholesterol content by HPLC.

### Analysis of Cordycepin by HPLC

Cordycepin used for standard was obtained from Sigma Chemical Company. For determination of cordycepin in eggs, the soft matter from 8 eggs were combined and the weight was measured. The combination was then homogenised. A 10 g sample of the homogenate was taken and extracted three times with 100 ml of hexane. The extract was collected and sonicated for 1.5 hours at 60 degrees. The sonicate was then filtered with 0.45 micron microsyringe filters before HPLC. The HPLC analysis was performed on a Shimadzu LC20A R1.24 employing 254 nm detection. 5 μl of the filtered sonicate was injected into a 0.45 microsyringe filter μL loop and chromatographed through a C18, 300×4.6 mm, 5 μm particle size, 300 Å column (Waters). The mobile phase for elution was acetonitrile-water (V/V 496) with phosphate buffer (Na₂PO₄, 0.1 M, pH 6.8) at a flow rate of 1.0 ml/min. The analysis was performed using the Shimadzu LCsolution software.

### Analysis of Cholesterol Content by HPLC

The HPLC analysis of cholesterol content of the eggs was performed, according to standard methodology, by TÜV SÜD, PSB Corporation, Singapore.

### Results

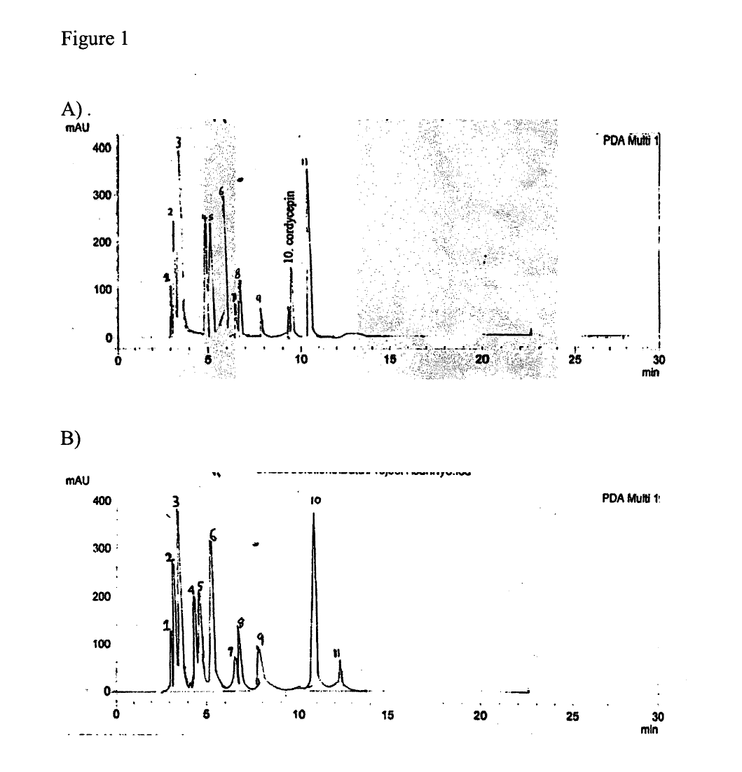

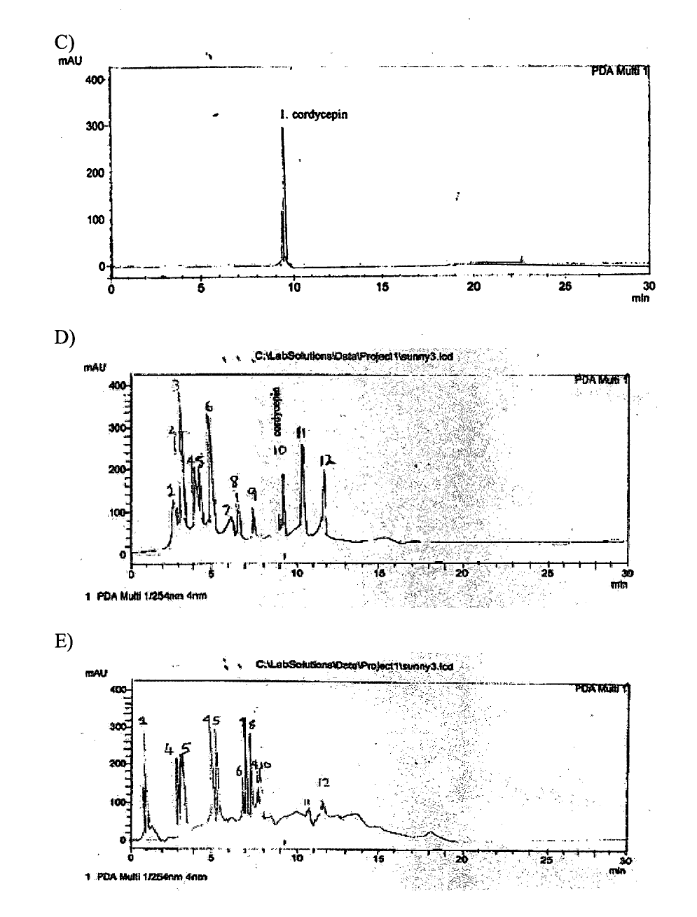

Cordycepin was detected in the eggs from layers fed with the animal feed comprising Cordyceps sinensis (FIG. 1A) and not in the eggs from control layers (FIG. 1B). Furthermore, the cordycepin was found predominantly in the egg yoke (FIG. 1D). Cordycepin was not detected in the egg white.

Table 10 shows the cordycepin and cholesterol contents of the eggs produced by the layers fed with the animal feed comprising the Cordyceps sinensis extract.

TABLE 10
Cordycepin and Cholesterol Content of eggs produced by layers
fed with animal feed comprising Cordyceps sinensis extract

| Addition of CS | | Cordycepin, | Cholesterol, |
|---|---|---|---|
| powder. | Feeding | Gram per 100 | Milligram per 100 |
| Gram per ton of | time | gram of egg matter | gram of egg matter |
| chicken layer feed, | weeks | (without shell) | (without shell) |
| 300 g | 8 | 0.043-0.08 | 202 |
| 50 g | 12 | 0.006-0.007 | 237 |
| 125 g | 10 | 0.02-004 | 293 |

The cordycepin levels in the eggs from the layers fed with the feed comprising the Cordyceps sinensis extract were measured and were found to be in the range of 0.006-0.08 g per g of whole egg and is dependent on the composition of Cordyceps extract in the feed. Similarly, the cholesterol content of the eggs produced were measured and ranged from 202 to 207 mg per 100 g of egg matter. As discussed above, normal eggs have a higher cholesterol content at 368.2±22.54 to 437.63±24.61 mg per 100 g of egg matter (Vorlová et al., 2001).

While there has been described in the foregoing description preferred embodiments of the present invention, it will be understood by those skilled in the technology concerned that many variations or modifications in details of design, construction or operation may be made without departing from the scope of the present invention as claimed.

### Example 6

### Effect of Cordyceps sinensis Mycelium Powder on Sprague Dawley Rats

10 male and 10 female Sprague Dawley (SD) rats, 6-8 weeks old were studied. The rats were fed on a conventional laboratory diet with unlimited supply of water. Before the test substance was administered orally, feed was withheld overnight. Body weights were recorded just before dosing (Day 1).

A test group comprising 5 male and 5 female rats were dosed by gavage with a 2 g/20 ml suspension of Cordyceps sinensis mycelium prepared by the method described in SG 200400686-2 (SG patent 129276), to a dose equivalent of 2 g/kg rat. The other 5 males and 5 females were dosed with water at 20 ml/kg rat. The rats were monitored for 15 days and the body weight was studied.

The body weight of the rats was monitored at days 2, 3, 4, 7, 10, 13 and 15. Table 11 shows the mean weight of the control group and the test group

TABLE 11

| Day | Mean weight/g (Control group) | Mean weight/g (Test group) |
|---|---|---|
| Day 1 | 193.7 | 195.4 |
| Day 2 | 213.18 | 214.6 |
| Day 3 | 220.52 | 221.6 |
| Day 4 | 227.08 | 228 |
| Day 7 | 245.3 | 249.1 |
| Day 10 | 260.6 | 267.3 |
| Day 13 | 278.2 | 285.1 |
| Day 15 | 285.4 | 297 |

This study suggests that rats which were administered with freeze-dried Cordyceps sinensis mycelium have a higher body weight gain compared to the rats in the control group.

Mortality: none of the rats died during the trial. From this study, the LD₅₀ for the freeze-dried Cordyceps sinensis mycelium is considered to be greater than 2 g/kg body weight.

### Example 7

### Feeding of Broiler Chickens with Cordyceps sinensis Mycelium

Two breeds of broiler chickens, a traditional breed, Three-Yellow from China and a fast growing breed, Cobb-500 were studied for growth performance and meat quality on feeding with Cordyceps sinensis mycelium powder.

### Example 7A

### Three-Yellow Chickens

Three-Yellow chickens are a common Chinese breed, named for their distinctive, yellow beak, yellow feat and yellow feathers. This traditional breed matures in 100-180 days.

In the study, 2000 Three-Yellow chickens were studied. The chickens were divided into two groups of 1000 chickens each. One group (the test group) was given commercial feed supplemented with Codyceps sinensis mycelium powder and the second group (control) was given unsupplemented feed for 100 days. The commercial feed was the Jiale brand obtained from Guangzhou Jiujiang Feed Co, Ltd and comprises 72% carbohydrate/fat, 16% protein, 0.5-0.8% total phosphorus, >0.3% methionine, 4% crude fiber, 0.7-1.2% calcium, 0.3-0.8% salt and 8% ash.

The test group was given the commercial feed supplemented with increasing amounts of Cordyceps sinensis mycelium powder prepared by the method described in SG 200400686-2 (SG patent 129276) as follows:

Day 1 to Day 10: 250 g Cordyceps sinensis mycelium powder per ton of feed.

Day 11 to Day 20: 500 g Cordyceps sinensis mycelium powder per ton of feed.

Day 21 to Day 60: 1000 g Cordyceps sinensis mycelium powder per ton of feed.

Day 61 to Day 100: 2000 g Cordyceps sinensis mycelium powder per ton of feed.

The following were observed in the two groups of chickens.

### Growth Performance

The mean weight of the 1000 Three-Yellow chickens in the test group and the control group was determined. The feed conversion ratio, defined as the mass of the feed consumed divided by the body mass gain, over a specified period of time (in this case, 100 days) was determined. These results are shown in Table 12.

TABLE 12
Mean weight and feed conversion ratio of Three-Yellow chickens
from the test group and control group

| | Test group | Control group |
|---|---|---|
| Average weight (kg) | 2.8 | 2.7 |
| Feed conversion ratio | 3.1 | 3.5 |
| (FCR) | | |

The test group of Three-Yellow chickens had an average weight slightly higher than the control group (consistent with the study on rats in Example 6). In addition, the test group had a slightly lower feed conversation ratio than the control group, which suggest that the test group are more efficient users of feed.

### Comparison of Meat Quality

The meat of 5 Three-Yellow chickens from the test group were pooled and analysed for protein, fat and amino acid content. The same analysis was performed on the pooled meat of 5 Three-Yellow chickens from the control group (Table 12).

Analysis of the protein, fat and amino acid content in the meat were performed by standard methods by the testing laboratory from Guangzhou Agricultural Standard and Supervisory Center.

TABLE 13
Comparsion of protein, fat and amino acid content of meat from
Three-Yellow chickens from the test group and control group

| | | | Total 16 major |
|---|---|---|---|
| | Protein, % | Fat, % | amino acids, % |
| Test group | 23.5 | 1.5 | 21.27 |
| Control group | 23.1 | 0.8 | 21.17 |

The results in Table 13 indicate that the protein and amino acid content increased marginally in the test group compared to the control, but the fat content was doubled. It is suggested that increased fat content may provide a better flavour and texture to the meat of Three-Yellow chickens.

### Appearance of the Chickens

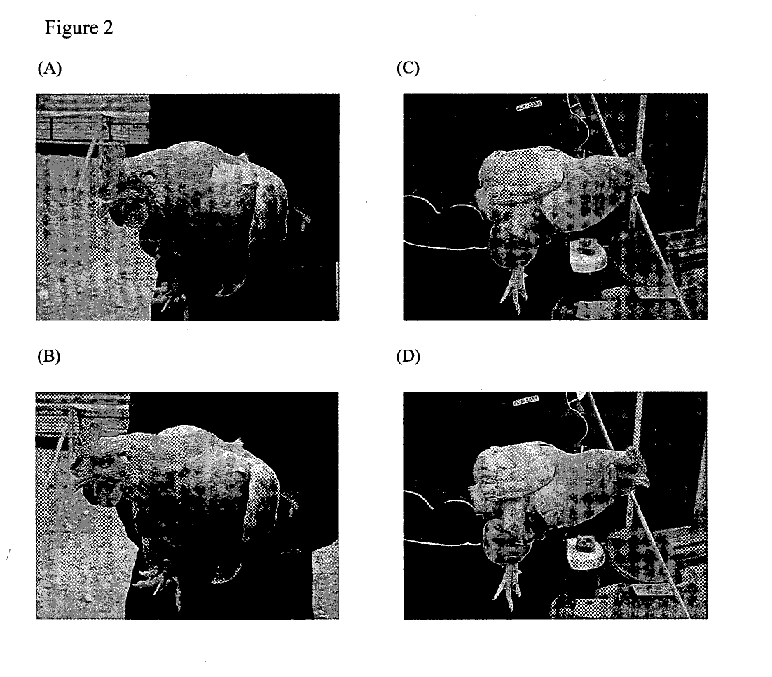

The Three-Yellow chickens fed with feed comprising Cordyceps sinensis mycelium powder had a better appearance than the control group as shown in FIG. 2. The combs of these chickens were a brighter red, their feathers were glossier and shinier and the beak, feathers and feet were a brighter yellow. In China, this better appearance of the Three-Yellow chickens will translate to a higher price and better sales performers due to consumer preference.

### Taste Test

A panel of five members unaminously agreed that the meat from the Three-yellow chicken whose diet was supplemented with Cordyceps sinensis mycelium powder tasted better than the meat from the control group.

Disease Prevention and/or Reduction

### (I) Chicken Coccidiosis

Chicken coccidiosis is a parasitic disease caused by protozoan of the Eimera genus. The parasite lives and multiplies in the intestinal tract and causes tissue damage. The incidence rate of clinical coccidiosis is estimated to be 5% while that of subclinical coccidiosis is estimated to be about 20%. Coccidiosis is estimated to cost the world's commercial chicken producers at least US$800 million dollars every year (Williams, 1998). This estimate is based on the costs of prophylactic in-feed medication, therapy and losses due to mortality, morbidity, and poor performance of surviving birds.

In this study, the group of Three-Yellow chickens whose feed was supplemented with Cordyceps sinensis mycelium powder exhibited a zero incidence of chicken coccidiosis. Comparatively, the control group repeatedly encountered 23% incidence of chicken coccidiosis and were treated with sulfadimidine to control the infection. The full course of treatment comprises 5 kg sulfadimidine for every 1000 kg of feed for three days, an intervening two day break comprising normal feed and another three days of 5 kg sulfadimidine for every 1000 kg of feed. Treatment with sulfadimidine for the control group on normal feed was used 26 times compared to the test group which did not require any treatment because no incidence of chicken coccidiosis was observed.

### (II) Infectious Bronchitis

Infectious bronchitis is another common disease in chicken farming, and the symptoms of this disease include tracheal rale, cough, sneexing and other respiratory symptoms. In this study, the test group whose feed was supplemented with Cordyceps sinensis mycelium powder was found to have a zero incidence of infectious bronchitis. Comparatively, isolated cases of tracheal rale, cough and sneezing were observed in the control group.

The zero incidence of chicken coccidiosis and infections bronchitis in the test group suggests that the Cordyceps sinensis mycelium powder is important in enhancing the immunity and resistance of the chickens in the test group to diseases.

### (III) Mortality

In this study, 30 chickens from the control group died while 29 chickens from the test group died. It is noted that although the mortality between the two groups were similar, the control group were provided with drugs to prevent coccidiosis, which may have enhanced their survival through improved disease resistance, not only to coccidiosis but also other diseases.

### Example 7B Cobb 500

Cobb 500 is a fast growing broiler breed which matures in 30-45 days. Two groups, each comprising 100 Cobb-500 chickens were used for the trial. One group was given commercial feed supplemented with Cordyceps sinensis mycelium powder at the amount of 100 g per 1000 kg of feed from hatching for 36 days. The control group was given the commercial feed only for the same 36 days.

### Analysis of Inosine Monophosphate Content.

The meat from two Cobb 500 chickens from the test group and one chicken from the control group were analysed for inosine monophosphate content. The inosine monophosphate (IMP) content of breast meat was determined as described in Vani et al., (2006). The IMP levels of the two chickens from the test group were 2.31 mg/g and 2.41 mg/g respectively. The IMP level of the control chicken was 1.61 mg/g. This IMP level from the control chicken is comparable to the 1.885 mg/g from breast meat reported in Vani et al., (2006).

The IMP content of is positively associated with the flavour of the meat (Vani et al., 2006). The higher IMP content of the test group compared to the control group suggests that meat from the test group has an improved flavour compared to the control group.

A taste test was also conducted on the Cobb 500 chicken by a five member panel, all of which agreed that the meat from the test group had better taste and flavour and had a smoother texture than that of the control group.

### REFERENCES

Siev M., Weinberg, R and Penman, S. (1969) The selective interruption of nucleolar RNA synthesis in HELA cells by cordycepin. J. Cell Biol., vol 41, 510-520.
Vani, N. D., Modi, V. K., Kavitha, S., Sachindra, N. M. and Mahendrakar, N. S. (2006) Degradation of inosine-5′-monophosphate (IMP) in aqueous and in layering chicken muscle fibre systems: Effect of pH and temperature. LWT Food Science and Technology, vol 39, 627-632.
Vorlová L., Sieglová E., Karpí\check{s}ková R. and Kop\check{r}iva V. (2001) Cholesterol content in eggs during the laying period. Acta Vet. Brno, vol 70, 387-390.
Williams, R. B. (1998) Epidemiological aspects of the use of live anticoccidial vaccines for chickens. Int J. Parasitol., vol 28, 1089-1098.
SG 200400686-2 (SG patent 129276)

1. A method of producing at least one egg comprising
(i) supplementing the diet of at least one animal with at least one Cordyceps species or extract thereof, or
(ii) feeding at least one animal with an animal feed comprising at least one feed ingredient and at least one Cordyceps species or extract thereof.

2. The method according to claim 1, wherein the Cordyceps species is selected from the group consisting of Cordyceps sinensis, Cordyceps militaris, Cordyceps gracilis, Cordyceps ophioglossoides, Cordyceps memorabiffis, Cordyceps sphecocephala, Cordyceps hawkesii, Cordyceps martialis, Cordyceps sobolifera and Cordyceps capitata

3. The method according to claim 1 or 2, wherein the Cordyceps species is Cordyceps sinensis .

4. The method according to any one of claims 1 to 3, wherein the egg comprises cordycepin.

5. The method according to claim 4, wherein the egg comprises 0.006-0.08 g cordycepin per 100 g of egg matter.

6. The method according to claim 4 or 5, wherein the cordycepin is predominantly in the egg yolk.

7. The method according to any one of claims 1 to 6, wherein the egg comprises reduced cholesterol content compared to at least one egg not produced by the method according to any one of claims 1 to 6.

8. The method according to any one of claims 1 to 7, wherein the egg comprises less than 350 mg cholesterol per 100 g of egg matter.

9. The method according to any of claims 1 to 8, wherein the egg comprises less than 300 mg cholesterol per 100 g of egg matter.

10. An egg produced by the method according to any one of claims 1 to 9.

11. An egg comprising cordycepin.

12. The egg according to claim 11 comprising 0.006-0.008 g cordycepin per 100 g of egg matter.

13. The egg according to claim 11 or 12, wherein the cordycepin is predominantly in the egg yolk.

14. The egg according to any one of claims 11 to 13, wherein the egg comprises less than 350 mg cholesterol per 100 g of egg matter.

15. The egg according to any of claims 11 to 14, wherein the egg comprises less than 300 mg cholesterol per 100 g of egg matter.

16. An egg comprising less than 350 mg cholesterol per 100 g of egg matter.

17. An egg comprising less than 300 mg cholesterol per 100 g of egg matter.

18. An egg according to any one of claims 10 to 17, for use in treating and/or prophylaxis of at least one disease.

19. The egg according to claim 18, wherein the disease is selected from the group consisting of respiratory disease, kidney disease, renal disease, diabetes, cancer, tumour and infectious disease.

20. A method of treating and/or prophylaxis of at least one disease comprising administering
(i) at least one egg according to any one of claims 10 to 18, or
(ii) an egg extract or egg product from at least one egg according to any one of claims 10 to 18 to a subject.

21. The method according to claim 20, wherein the disease is selected from the group consisting of respiratory disease, kidney disease, renal disease, diabetes, cancer, tumour and infectious disease.

22. Use of at least one egg according to any one of claims 10 to 18, or an egg extract or egg product therefrom for the preparation of a composition for the treatment and/or prophylaxis at least one disease.

23. The use according to claim 22 wherein the disease is selected from the group consisting of respiratory disease, kidney disease, renal disease, diabetes, cancer, tumour and infectious disease.

24. Use of at least one Cordyceps species or extract thereof in the preparation of a dietary supplement for producing at least one egg.

25. The use according to claim 24, wherein the Cordyceps species is selected from the group consisting of Cordyceps sinensis, Cordyceps militaris, Cordyceps gracilis, Cordyceps ophioglossoides, Cordyceps memorabiffis Cordyceps sphecocephala, Cordyceps hawkesii, Cordyceps martialis, Cordyceps sobolifera and Cordyceps capitata.

26. The use according to claim 24 or 25, wherein the Cordyceps species is Cordyceps sinensis.

27. The use according to any one of claims 24 to 26, wherein the egg comprises cordycepin.

28. The use according to claim 27, wherein the egg comprises 0.006 to 0.08 mg cordycepin per 100 g of egg matter.

29. The use according to any one of claims 27 to 28, wherein the cordycepin is predominantly in the egg yolk.

30. The use according to any of claims 24 to 29, wherein the egg comprises less than 350 mg cholesterol per 100 g of egg matter.

31. The use according to any of claims 24 to 30, wherein the egg comprises less than 300 mg cholesterol per 100 g of egg matter.

32. A dietary supplement comprising an extract of at least one Cordyceps species.

33. The dietary supplement according to claim 32, wherein the Cordyceps species is selected from the group consisting of Cordyceps sinensis, Cordyceps militaris, Cordyceps gracilis, Cordyceps ophioglossoides, Cordyceps memorabiffis Cordyceps sphecocephala, Cordyceps hawkesii, Cordyceps martialis, Cordyceps sobolifera and Cordyceps capitata.

34. The dietary supplement according to claim 32 or 33, wherein the Cordyceps species is Cordyceps sinensis.

35. An animal feed comprising at least one feed ingredient and a dietary supplement according to any one of claims 32 to 34

36. A method of supplementing the diet of animals, comprising
(i) providing the dietary supplement according to any one of claims 32 to 34 to at least one animal, and/or
(ii) feeding at least one animal with an animal feed according to claim 35.

37. The method according to claim 36, wherein providing the dietary supplement comprises mixing the dietary supplement with the feed and providing the mixture to the animal.

38. The method according to claim 36 or 37, wherein the animal is an oviparous animal.

39. The method according to claim 38, wherein the oviparous animal is a poultry.

40. The method according to claim 39, wherein the poultry is a fowl.

41. The method according to any one of claims 38 to 40, for producing at least one egg.

42. The method according to claim 41, wherein the egg comprises cordycepin.

43. The method according to claim 42, wherein the egg comprises 0.006-0.08 mg cordycepin per 100 g of egg matter.

44. The method according to claim 42 or 43, wherein the cordycepin is predominantly in the egg yolk.

45. The method according to any one of claims 41 to 44, wherein the egg comprises less than 350 mg cholesterol per 100 g of egg matter.

46. The method according to any one of claims 41 to 45, wherein the egg comprises less than 300 mg cholesterol per 100 g of egg matter.

47. An animal whose diet is supplemented according to the method of any one of claims 36 to 46.

48. The animal according to claim 47, wherein the animal is oviparous.

49. The animal according to claim 48, wherein the animal is poultry.

50. The animal according to claim 47 or 48, wherein the animal is fowl.

51. An egg obtainable from the method of any one of claims 41 to 46.

52. An animal according to any one of claims 47-50 for use in producing meat.

53. Meat from the animal according to any one of claims 47-50 and 52.
